(19) United States
(12) Patent Application Publication
(10) Pub. No.: US 2009/0162897 A1
(43) Pub. Date: Jun. 25, 2009

(54) Promoter Sequences

(75) Inventors: Heiko Eichhorn, Kundl (AT); Thomas Specht, Kundl (AT); Ivo Zadra, Heufeld (DE)

Correspondence Address: Mark S. Graham, Esq.;LUEDEKA, NEELY & GRAHAM, P.C., P.O. Box 1871, Knoxville, TN, 37901, US

(73) Assignee: SANDOZ AG, Basel, CH (CH)

(21) Appl. No.: 12/097,799

(22) PCT Filed: Dec. 20, 2006
(86) PCT No.: PCT/EP2006/012319

§ 371 Date: Jun. 17, 2008

(30) Foreign Application Priority Data
Dec. 22, 2005 (EP) 05028139.3

(51) Int. Cl. C12P 21/00; C12N 15/11; C12N 15/80; C12N 1/15; C07H 21/02; C12P 19/34; C12P 1/02

(52) U.S. Cl. 435/69.1; 536/23.1; 435/320.1; 435/254.11; 536/24.5; 536/24.1; 435/91.1; 435/254.3; 435/254.5; 435/41

(57) ABSTRACT

The present invention relates to methods for producing polypeptides or nucleic acids, in particular antisense RNA and hairpin RNA in filamentous fungi. The present invention also relates to isolated Penicillium promoter sequences and to nucleic acid constructs, vectors, and host cells comprising the promoter sequences operably linked to nucleic acid sequences encoding polypeptides or nucleic acids, in particular antisense RNA and hairpin RNA. Furthermore, the present invention relates to fermentative production of small organic compounds by use of the Penicillium promoter sequences.

### FIELD OF THE INVENTION

The present invention relates to methods for producing polypeptides or nucleic acids, in particular antisense RNA and hairpin RNA in filamentous fungi. The present invention also relates to isolated Penicillium promoter sequences and to nucleic acid constructs, vectors, and host cells comprising the promoter sequences operably linked to nucleic acid sequences encoding polypeptides or nucleic acids, in particular antisense RNA and hairpin RNA. Furthermore, the present invention relates to the use of the Penicillium promoter sequences for fermentative production of small organic compounds.

### BACKGROUND OF THE INVENTION

The recombinant production of homologous and heterologous proteins in a fungal host cell, particularly a filamentous fungal cell such as Penicillium allows production of proteins in commercially relevant quantities. Furthermore overexpression of homologous and heterologous proteins or expression of antisense RNA or hairpin RNA allows to increase the production of small organic compounds like β-lactam antibiotics such as penicillin and to reduce undesired by-products.

Recombinant production of a protein or of nucleic acids such as antisense RNA and hairpin RNA is accomplished by constructing an expression vector in which the DNA coding for the protein or the nucleic acid is placed under the expression control of a promoter, excised from a regulated gene, suitable for the host cell. The expression vector is introduced into the host cell, usually by plasmid-mediated transformation. Production of the protein or of the nucleic acid is then achieved by culturing the transformed host cell under suitable conditions necessary for the proper functioning of the promoter contained in the expression vector.

The filamentous fungi Penicillium chrysogenum and Acremonium chrysogenum have the ability to produce antibiotics like penicillin and cephalosporin and are therefore of industrial interest. In order to increase productivity of commercially used production strains techniques for genetic manipulation of these strains have been developed in the last couple of years. These techniques include the transformation of protoplasts with vectors including a selection marker like the phleomycin resistance gene (Kolar, M. et al. (1988), Gene 62, 127-134). The alteration of the expression of genes of interest for example due to the replacement of the promoters of these genes by other homologous or heterologous promoters in order to improve gene expression has been shown. Especially for very poorly expressed genes for example for genes such as the cefG gene in Acremonium chrysogenum, the utilization of promoters of constitutively highly expressed genes has been reported to have a positive impact on fermentation titers and by product profile of cephalosporin (Gutierrez S., et al. (1997) Applied Microbiology and Biotechnology 48(5), 606-614).

The inactivation of gene expression, either partially or almost completely, is used to eliminate or at least down-regulate activities of enzymes or regulators in order to eliminate undesirable regulatory pathways or regulators in a specific signaling cascade. In most cases gene disruption is not appropriate because of the level of ploidy or because it results in a non-viable organism.

The use of antisense constructs to modulate gene regulation or blocking enzyme activities has been shown in Penicillium chrysogenum, Aspergillus nidulans and several other filamentous fungi as a valuable approach, in particularly if loss-of-function mutations exhibit impaired growth parameters making this strategy inapplicable for industrial purposes (Zadra et al. (2000), Appl. Environ. Microbiol. 66, 4810-4816; Bautista, L. F. et al. (2000), Appl. Environ. Microbiol. 66(10), 4579-4581 and Wang, T. H. et al. (2005), Lett. Appl. Microbiol. 40, 424-429.)

In addition to the well known antisense technology, RNA-mediated gene silencing, a new method to alter gene expression, is extensively characterized in various model organisms from plants to animals. Recently this technology is also established in filamentous fungi. The RNA-mediated gene silencing is a posttranscriptional gene-silencing in which double-stranded RNA (dsRNA) triggers the degradation of cognate mRNA in a sequence-specific manner (Nakayashiki, H. et al. (2005), Fungal Genetics and Biology 42, 275-283). Despite the proof that RNA-mediated gene silencing is functional in several fungal species (Nakayashiki, H. et al. (2005), Fungal Genetics and Biology 42, 275-283; Kadotani, N. et al. (2003) Mol. Plant-Microbiol. Interactions 16, 769-776 and Mouyna, I. et al. (2004), FEMS Microbiol. Lett. 237, 317-324) for filamentous fungi this technology lies only in the beginnings. This is mainly due to limited genetic tools available, e.g. only a limited number of silencing vectors suitable for fungal applications and/or for generating hairpin constructs for stable transformations is available. Furthermore, it is reported that the choice of the promoter as well as of the internal spacer region in such a vector has a strong impact on the efficiency on RNA-mediated gene silencing, also called RNA interference, in filamentous fungi. (Nakayashiki, H. et al. (2005), Fungal Genetics and Biology 42, 275-283). There seems to be, however, a lack of such promoters suitable for use in those vectors.

There are citations in the prior art describing the use of promoters from several housekeeping genes from different filamentous fungi for the expression or inactivation of gene expression. It could be shown, however, that some housekeeping genes like the Aspergillus nidulans gpdA gene (encoding glyceraldehyde-3-phosphate dehydrogenase) or the γ(gamma)-actin gene the promoters of which are frequently used for over-expression or inactivation of gene expression are expressed in a developmentally regulated manner or in a rhythmically regulated manner (Greene, A. V. et al. (2003), Eukaryotic Cell 2(2), 231-237 and Jeong, H-Y. et al. (2001), Gene 262 (1-2), 215-219).

Therefore, there is a need in the art for new promoters for controlling gene expression in filamentous fungi, in particular for new promoters showing a constitutive stable and high expression and being furthermore suitable for use in the above described methods, e.g. in the vectors suitable for RNA-mediated gene silencing.

### SUMMARY OF THE INVENTION

The present invention therefore provides improved methods for producing a polypeptide or a nucleic acid, preferably an antisense RNA or a hairpin RNA in a fungal host cell and new promoters for such production. The new promoters of the invention show a constitutive, stable, high transcription rate under conditions of fermentation, and are therefore suitable for use in the production of small organic compounds obtainable by fermentation and/or for manipulating the metabolism of such a fungal host cell. Additionally, the new promoters of the invention are suitable for manipulating growth and/or pathways involved in regulation of morphology and/or sporulation of such a fungal host cell. Furthermore the promoters of the invention are suitable for the herein mentioned method of RNA-mediated gene silencing.

Thus, the present invention relates to a method for producing a polypeptide or a nucleic acid, preferably an antisense RNA or a hairpin RNA, comprising the steps of:

(a) cultivating a fungal host cell in a medium conductive for the production of the polypeptide or the nucleic acid, preferably the antisense RNA or the hairpin RNA, wherein the fungal host cell comprises a first nucleic acid sequence encoding the polypeptide or the nucleic acid, preferably the antisense RNA or the hairpin RNA, operably linked to a second nucleic acid sequence comprising a promoter sequence selected from the group consisting of SEQ ID NO. 4, SEQ ID NO. 2, SEQ ID NO. 3, SEQ ID NO. 5, SEQ ID NO. 1, and subsequences thereof wherein said subsequences are at least 100 nucleotides, and mutant, hybrid, and tandem promoters thereof wherein said mutant has at least about 20% of the promoter activity of said promoter sequences; and
(b) isolating the polypeptide from the culture medium, or
(c) isolating the polypeptide from the fungal host cell.

The present invention also relates to isolated nucleic acid sequences comprising a nucleic acid sequence selected from the group consisting of SEQ ID NO. 4, SEQ ID NO. 2, SEQ ID NO. 3, SEQ ID NO. 5, SEQ ID NO. 1 and subsequences thereof wherein said subsequences are at least 100 nucleotides.

The present invention also relates to isolated nucleic acid sequences, selected from the group consisting of

(a) a nucleic acid sequence having at least 80% homology with a sequence selected from the group consisting of SEQ ID NO. 4, SEQ ID NO. 2, SEQ ID NO. 3, SEQ ID NO. 5 and SEQ ID NO. 1;
(b) a nucleic acid sequence which hybridizes under conditions of stringency with (i) a sequence selected from the group consisting of SEQ ID NO. 4, SEQ ID NO. 2, SEQ ID NO. 3, SEQ ID NO. 5 and SEQ ID NO. 1; (ii) a subsequence of (i) of at least 100 nucleotides, or (iii) a complementary strand of (i) or (ii);
(c) a nucleic acid sequence of (a) or (b) comprising a substitution, deletion, and/or insertion of one or more nucleotides;
(d) an allelic variant of (a), (b), or (c); and
(e) a subsequence of (a), (b), (c), or (d),
wherein the conditions of stringency of (b) are defined as a series of washes starting with 6×SSC, 0.5% SDS at room temperature for 15 min, then repeated with 2×SSC, 0.5% SDS at 45° C. for 30 min, and then repeated twice with 0.2×SSC, 0.5% SDS at 60° C.

The present invention also relates to nucleic acid constructs comprising a nucleic acid sequence encoding a polypeptide or a nucleic acid, preferably an antisense RNA or a hairpin RNA, operably linked to the nucleic acid sequence described above. It further relates to recombinant expression vectors and a recombinant host cells comprising the nucleic acid constructs.

Furthermore, the present invention provides a method for fermentative production of a small organic compound, which small organic compound is obtainable by fermentative production, comprising the steps of:

(a) cultivating a fungal host cell in a medium conductive for the production of a polypeptide or of a nucleic acid, preferably an antisense RNA or a hairpin RNA, which are directed to regulate a metabolic pathway of said host cell thereby leading to the production of said small organic compound, wherein the fungal host cell comprises a first nucleic acid sequence encoding the polypeptide or the nucleic acid, preferably the antisense RNA or the hairpin RNA, operably linked to a second nucleic acid sequence comprising a promoter sequence selected from the group consisting of SEQ ID NO. 4, SEQ ID NO. 2, SEQ ID NO. 3, SEQ ID NO. 5, SEQ ID NO. 1, and subsequences thereof wherein said subsequences are at least 100 nucleotides, and mutant, hybrid, and tandem promoters thereof wherein said mutant has at least about 20% of the promoter activity of said promoter sequences; and
(b) allowing said polypeptide or antisense or hairpin RNA to be expressed; and
(c) isolating the small organic compound from the fermentation broth.

Additionally, the present invention relates to the use of a nucleic acid sequence of the invention for the production of a small organic compound by fermenative production and/or for manipulating the metabolism and/or growth and/or pathways involved in regulation of morphology and/or sporulation of a filamentous fungal host cell.

### BRIEF DESCRIPTION OF THE FIGURES

FIG. 1 shows the Pc12g09320 promoter sequence (SEQ ID NO. 1)

FIG. 2 shows the Pc12g100000 promoter sequence (SEQ ID NO. 2)

FIG. 3 shows the Pc16g00660 promoter sequence (SEQ ID NO. 3)

FIG. 4 shows the Pc21g04830 promoter sequence (SEQ ID NO. 4)

FIG. 5 shows the Pc21g20300 promoter sequence (SEQ ID NO. 5)

### DETAILED DESCRIPTION OF THE INVENTION

The present invention relates to a method for producing a polypeptide or a nucleic acid, preferably an antisense RNA or a hairpin RNA, comprising the steps of:

(a) cultivating a fungal host cell in a medium conductive for the production of the polypeptide the nucleic acid, preferably the antisense RNA or the hairpin RNA, wherein the fungal host cell comprises a first nucleic acid sequence encoding the polypeptide or the nucleic acid, preferably the antisense RNA or the hairpin RNA, operably linked to a second nucleic acid sequence comprising a promoter sequence selected from the group consisting of SEQ ID NO. 1, SEQ ID NO. 2, SEQ ID NO. 3, SEQ ID NO. 4, SEQ ID NO. 5, and subsequences thereof, and mutant, hybrid, and tandem promoters thereof; and
(b) isolating the polypeptide from the culture medium, or
(c) isolating the polypeptide from the fungal host cell.

The subsequences mentioned under step (a) are preferably at least 100 nucleotides. The mutant promoters of step (a) may have at least about 20% of the promoter activity of the promoter sequences mentioned under step (a).

The terms “a medium conductive—or suitable—for the production of a polypeptide or a nucleic acid, preferably an antisense RNA or a hairpin RNA” are understood to mean a medium conductive—or suitable—for the proper functioning of the promoters of the invention which may be contained in a suitable expression vector as herein described to obtain the desired polypeptide or nucleic acid.

Preferably, the fungal host cell in step (a) and/or in step (c) is a filamentous fungal host cell. More preferably, the fungal host cell in step (a) and/or in step (c) is Penicillium chrysogenum or Acremonium chrysogenum or Aspergillus terreus or Penicillium citrinum.

Thus, present invention relates to a method for producing a polypeptide or a nucleic acid, preferably an antisense RNA or a hairpin RNA, comprising step (a) and step (b), or comprising step (a) and step (c) as described above, wherein the fungal host cell in step (a) and/or step (c) is a filamentous fungal host cell, preferably Penicillium chrysogenum or Acremonium chrysogenum or Aspergillus terreus or Penicillium citrinum.

In the production methods of the present invention, the cells are cultivated in a nutrient medium suitable for production of the polypeptide or of the nucleic acids, preferably the antisense RNA or the hairpin RNA using methods known in the art. For example, the cell may be cultivated by shake flask cultivation, small-scale or large-scale fermentation (including continuous, batch, fed-batch, or solid state fermentations) in laboratory or industrial fermentors performed in a suitable medium and under conditions allowing the polypeptide or the nucleic acid, in particular the antisense RNA or the hairpin RNA, to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g. in catalogues of the American Type Culture Collection, or e.g. as described by Gutierrez, S. et al. (1999), Microbiology 145, 317-324). If the polypeptide is secreted into the nutrient medium, the polypeptide can be recovered directly from the medium. If the polypeptide is not secreted, it can be recovered from cell lysates. The resulting polypeptide may be recovered by methods known in the art. For example, the polypeptide may be recovered from the nutrient medium by conventional procedures including, but not limited to, centrifugation, filtration, extraction, spray-drying, evaporation, or precipitation.

The polypeptides may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, J.-C. Janson and Lars Ryden, editors, VCH Publishers, New York, 1989).

The term “promoter” is defined herein as a DNA sequence that binds RNA polymerase and directs the polymerase to the correct downstream transcriptional start site of a nucleic acid sequence encoding a polypeptide or a nucleic acid such as an antisense RNA or a hairpin RNA, to initiate transcription. RNA polymerase effectively catalyzes the assembly of messenger RNA complementary to the appropriate DNA strand of the coding region. The term “promoter” will also be understood to include the 5′ non-coding region (between promoter and translation start) for translation after transcription into mRNA, cis-acting transcription control elements such as enhancers, and other nucleotide sequences capable of interacting with transcription factors.

The term “mutant promoter” is defined herein as a promoter having a nucleotide sequence comprising a substitution, deletion, and/or insertion of one or more nucleotides of a parent promoter, wherein the mutant promoter has more or less promoter activity than the corresponding parent promoter. The term “mutant promoter” will also encompass natural variants and in vitro generated variants obtained using methods well known in the art such as classical mutagenesis, site-directed mutagenesis, and DNA shuffling.

The term “hybrid promoter” is defined herein as parts of two or more promoters that are fused together to generate a sequence that is a fusion of the two or more promoters, which is operably linked to a coding sequence and mediates the transcription of the coding sequence into mRNA.

The term “tandem promoter” is defined herein as two or more promoter sequences each of which is operably linked to a coding sequence and mediates the transcription of the coding sequence into mRNA.

The term “operably linked” is defined herein as a configuration in which a control sequence, e.g., a promoter sequence, is appropriately placed at a position relative to a coding sequence such that the control sequence directs the production of a polypeptide or of a nucleic acid such as an antisense RNA or a hairpin RNA, encoded by the coding sequence. The term “coding sequence” is defined herein as a nucleic acid sequence that is transcribed into mRNA which is translated into a polypeptide when placed under the control of the appropriate control sequences. The boundaries of the coding sequence are generally determined by the ATG start codon located just upstream of the open reading frame at the 5′ end of the mRNA and a transcription terminator sequence located just downstream of the open reading frame at the 3′ end of the mRNA. A coding sequence can include, but is not limited to, genomic DNA, cDNA, semisynthetic, synthetic, and recombinant nucleic acid sequences. The coding sequence is also understood to encode a nucleic acid such as an antisense RNA or a hairpin RNA.

In a preferred embodiment of the method for producing a polypeptide or a nucleic acid such as an antisense RNA or a hairpin RNA as described above, the promoter has the nucleic acid sequence of SEQ ID NO. 1 or a subsequence thereof.

In another preferred embodiment, the promoter has the nucleic acid sequence of SEQ ID NO. 5 or a subsequence thereof.

In an even more preferred embodiment, the promoter has the nucleic acid sequence of SEQ ID NO. 3 or a subsequence thereof.

In another even more preferred embodiment, the promoter has the nucleic acid sequence of SEQ ID NO. 2 or a subsequence thereof.

In another still more preferred embodiment, the promoter has the nucleic acid sequence of SEQ ID NO. 4 or a subsequence thereof.

In the methods of the present invention, the promoter may also be a mutant of the promoters described above having a substitution, deletion, and/or insertion of one or more nucleotides. A mutant promoter may have one or more mutations. Each mutation is an independent substitution, deletion, and/or insertion of a nucleotide. The introduction of a substitution, deletion, and/or insertion of a nucleotide into the promoter may be accomplished using any of the methods known in the art such as classical mutagenesis, site-directed mutagenesis, or DNA shuffling.

In the methods of the present invention, the promoter may also be a hybrid promoter comprising a portion of one or more promoters of the present invention; a portion of a promoter of the present invention and a portion of another promoter, e.g., a leader sequence of one promoter and the transcription start site from the other promoter; or a portion of one or more promoters of the present invention and a portion of one or more other promoters. The other promoter may be any promoter sequence which shows transcriptional activity in the fungal host cell of choice including a mutant, truncated, and hybrid promoter, and may be obtained from genes encoding extracellular or intracellular polypeptides either homologous or heterologous to the host cell. The other promoter sequence may be native or foreign to the nucleic acid sequence encoding the polypeptide and native or foreign to the cell. Examples of other promoters useful in the construction of hybrid promoters with the promoters of the present invention include the promoters obtained from the genes which are regulated, for example induced or repressed by any compounds of the culture medium, like carbohydrates, nitrogen, phosphate, sulphur etc. or any chemical inductor known in the art. Promoters useful in the construction of hybrid promoters with the promoters of the present invention may also be induced or repressed by any physical condition, for example temperature.

The promoter may also be a tandem promoter comprising two more promoters of the present invention or alternatively one or more promoters of the present invention and one or more other promoters, such as those exemplified above. The two or more promoter sequences of the tandem promoter may simultaneously promote the transcription of the nucleic acid sequence. Alternatively, one or more of the promoter sequences of the tandem promoter may promote the transcription of the nucleic acid sequence at different stages of growth of the cell.

A mutant, hybrid, or tandem promoter of the present invention has at least about 20%, preferably at least about 40%, more preferably at least about 60%, more preferably at least about 80%, more preferably at least about 90%, more preferably at least about 100%, even more preferably at least about 200%, most preferably at least about 300%, and even most preferably at least about 400% of the promoter activity of the promoter of SEQ ID NO. 1, SEQ ID NO. 2, SEQ ID NO. 3, SEQ ID NO. 4 or SEQ ID NO. 5.

The polypeptide encoded by the nucleic acid sequence may be native or heterologous to the fungal host cell of interest.

The term “polypeptide” is not meant herein to refer to a specific length of the encoded product and, therefore, encompasses peptides, oligopeptides, and proteins. The term “heterologous polypeptide” is defined herein as a polypeptide which is not native to the fungal cell, a native polypeptide in which modifications have been made to alter the native sequence, or a native polypeptide whose expression is quantitatively altered as a result of a manipulation of the fungal cell by recombinant DNA techniques. For example, a native polypeptide may be recombinantly produced by, e.g., placing a gene encoding the polypeptide under the control of a promoter of the present invention to enhance expression of the polypeptide, to expedite export of a native polypeptide of interest outside the cell by use of a signal sequence, and to increase the copy number of a gene encoding the polypeptide normally produced by the cell. The fungal cell may contain one or more copies of the nucleic acid sequence encoding the polypeptide. The native or homologue polypeptide as well as the heterologous polypeptide may be either a non-secreted or a secreted polypeptide.

Preferably, the polypeptide is a native or homologue polypeptide. In a preferred embodiment, the polypeptide is a non-secreted polypeptide.

The term “antisense RNA” is defined herein as being an RNA complementary to the mRNA of a gene of interest, e.g. encoding a polypeptide of interest as herein described; such antisense RNA are described e.g. in Cooper G. M., The Cell. A Molecular Approach, 2^{nd} edition, 2000 (online version). Such antisense RNA may be obtained e.g. by a technique where a part of or a whole coding sequence of a homologous polypeptide is placed under the control of a promoter of the present invention in inverted direction in order to produce antisense RNA leading to down regulation of the expression of the native homologous polypeptide of interest. The fungal host cell may contain one or more copies of the nucleic acid sequence encoding the polypeptide.

The term “hairpin RNA” is defined herein as being an RNA having a hairpin structure such as described e.g. in Alberts B. et al., Molecular Biology of the Cell, 4^{th} Edition, 2002 (online version). Such hairpin RNA may be obtained by a technique where part of or a whole coding sequence of a homologous polypeptide is placed under the control of a promoter of the present invention in both sense and antisense orientation separated by a small internal spacer region in order to produce hairpin RNAs with double stranded regions which in turn are down regulating the expression of the native homologous polypeptide of interest by a complex cellular mechanism described in the literature as RNA interference. The fungal cell may contain one or more copies of the nucleic acid sequence encoding the polypeptide.

The present invention also relates to an isolated nucleic acid sequence comprising a nucleic acid sequence selected from the group consisting of SEQ ID NO. 4, SEQ ID NO. 2, SEQ ID NO. 3, SEQ ID NO. 5, SEQ ID NO. 1 and subsequences thereof.

In a preferred embodiment, the isolated nucleic acid sequence comprises the nucleic acid sequence of SEQ ID NO. 1 or a subsequence thereof.

In another preferred embodiment, the isolated nucleic acid sequence comprises the nucleic acid sequence of SEQ ID NO. 5 or a subsequence thereof.

In an even more preferred embodiment, the isolated nucleic acid sequence comprises the nucleic acid sequence of SEQ ID NO. 3 or a subsequence thereof.

In another even more preferred embodiment, the isolated nucleic acid sequence comprises the nucleic acid sequence of SEQ ID NO. 2 or a subsequence thereof.

In another still more preferred embodiment, the isolated nucleic acid sequence comprises the nucleic acid sequence of SEQ ID NO. 4 or a subsequence thereof.

The present invention also relates to nucleic acid constructs comprising a nucleic acid sequence encoding a polypeptide or a nucleic acid, preferably an antisense RNA or a hairpin RNA, operably linked to a promoter of the present invention and one or more control sequences which direct the expression of the coding sequence in a suitable host cell under conditions compatible with the control sequences.

The term nucleic acid construct is synonymous with the term expression cassette when the nucleic acid construct contains a coding sequence and all the control sequences required for expression of the coding sequence.

In the methods of the present invention, the nucleic acid sequence may comprise one or more native control sequences or one or more of the native control sequences may be replaced with one or more control sequences foreign to the nucleic acid sequence for improving expression of the coding sequence in a host cell.

The term “control sequences” is defined herein to include all components which are necessary or advantageous for the expression of a polypeptide or of a nucleic acid, preferably an of antisense RNA or of a hairpin RNA, according to the present invention. Each control sequence may be native or foreign to the nucleic acid sequence encoding the polypeptide or nucleic acid described above. Such control sequences include, but are not limited to, a leader, polyadenylation sequence, propeptide sequence, promoter of the present invention, signal peptide sequence, and transcription terminator. At a minimum, the control sequences include a promoter of the present invention, and transcriptional and translational stop signals. The control sequences may be provided with linkers for the purpose of introducing specific restriction sites facilitating ligation of the control sequences with the coding region of the nucleic acid sequence encoding a polypeptide or a nucleic acid, preferably an antisense RNA or a hairpin RNA.

The present invention also relates to recombinant expression vectors comprising a promoter of the present invention, a nucleic acid sequence encoding a polypeptide or of a nucleic acid, preferably of an antisense RNA or of a hairpin RNA, and transcriptional and translational stop signals. The various nucleic acid and control sequences described above may be joined together to produce a recombinant expression vector which may include one or more convenient restriction sites to allow for insertion or substitution of the promoter and/or nucleic acid sequence encoding the polypeptide or the nucleic acid, preferably an antisense RNA or a hairpin RNA, at such sites. Alternatively, the nucleic acid sequence may be expressed by inserting the nucleic acid sequence or a nucleic acid construct comprising the promoter and/or sequence into an appropriate vector for expression. In creating the expression vector, the coding sequence is located in the vector so that the coding sequence is operably linked with a promoter of the present invention and one or more appropriate control sequences for expression.

The recombinant expression vector may be any vector (e.g., a plasmid or virus) which can be conveniently subjected to recombinant DNA procedures and can bring about the expression of the nucleic acid sequence. The choice of the vector will typically depend on the compatibility of the vector with the host cell into which the vector is to be introduced. The vectors may be linear or closed circular plasmids.

The vector may be an autonomously replicating vector, i.e., a vector which exists as an extrachromosomal entity, the replication of which is independent of chromosomal replication, e.g., a plasmid, an extrachromosomal element, a minichromosome, or an artificial chromosome. The vector may contain any means for assuring self-replication.

Alternatively, the vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell, or a transposon may be used. The vector may be introduced into the host cell in combination with one or more additional vectors by the method of co-transformation.

The vectors of the present invention preferably contain one or more selectable markers which permit easy selection of transformed cells. A selectable marker is a gene the product of which provides for biocide or viral resistance, resistance to heavy metals, prototrophy to auxotrophs, and the like. Suitable markers for use in a filamentous fungal host cell include, but are not limited to, hygromycin or phleomycin. Further suitable markers are for example described in Finkistein, Ball (eds.), Applied Molecular Genetics of Filamentous fungi (ibid.) Biotechnology of Filamentous fungi, Butterworth-Heinemann, Boston, 1992.

The present invention also relates to recombinant host cells, comprising a promoter of the present invention operably linked to a nucleic acid sequence encoding a polypeptide or a nucleic acid, preferably an antisense RNA or a hairpin RNA, which are advantageously used in the recombinant production of the polypeptides or of the nucleic acids. A vector comprising a promoter of the present invention operably linked to a nucleic acid sequence encoding a polypeptide or a nucleic acid, preferably an antisense RNA or a hairpin RNA, is introduced into a host cell so that the vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the polypeptide and its source. Alternatively, the choice of the host cell may depend upon the nucleic acid sequence encoding the nucleic acid such as the antisense RNA or the hairpin RNA herein described.

The host cell may be any fungal cell useful in the methods of the present invention. In a preferred embodiment, the fungal host cell is a filamentous fungal cell. Preferably, the filamentous fungal cell is Penicillium chrysogenum or Acremonium chrysogenum or Aspergillus terreus or Penicillium citrinum.

The present invention also relates to isolated nucleic acid sequences, selected from the group consisting of

(a) a nucleic acid sequence having at least 80% homology with a sequence selected from the group consisting of SEQ ID NO. 4, SEQ ID NO. 2, SEQ ID NO. 3, SEQ ID NO. 5 and SEQ ID NO. 1;
(b) a nucleic acid sequence which hybridizes under conditions of stringency with (i) a sequence selected from the group consisting of SEQ ID NO. 4, SEQ ID NO. 2, SEQ ID NO. 3, SEQ ID NO. 5 and SEQ ID NO. 1; (ii) a subsequence of (i) of at least 100 nucleotides, or (iii) a complementary strand of (i) or (ii);
(c) a nucleic acid sequence comprising a substitution, deletion, and/or insertion of one or more nucleotides;
(d) an allelic variant of (a), (b), or (c); and
(e) a subsequence of (a), (b), (c), or (d).

The conditions of stringency of (b) may be defined as a series of washes starting with 6×SSC, 0.5% SDS at room temperature for 15 min, then repeated with 2×SSC, 0.5% SDS at 45° C. for 30 min, and then repeated twice with 0.2×SSC, 0.5% SDS at 60° C.

Preferably, the nucleic acid sequence of (c) is a nucleic acid sequence of (a) or (b).

In one embodiment, the present invention relates to isolated nucleic acid sequences having at least 80% homology with a sequence selected from the group consisting of SEQ ID NO. 4, SEQ ID NO. 2, SEQ ID NO. 3, SEQ ID NO. 5 and SEQ ID NO. 1.

Preferably, the degree of homology is about 85%, more preferably about 90%, even more preferably about 95%, and most preferably about 99%.

For purposes of the present invention, sequence comparisons are carried out using a Smith-Waterman sequence alignment algorithm (see e.g. Waterman, M. S. Introduction to Computational Biology Maps, sequences and genomes. Chapman & Hall. London: 1995. ISBN 0-412-99391-0, or at http://www-hto.usc.edu/software/segaln/index.html). The localS program, version 1.16, is used with following parameters: match: 1, mismatch penalty: 0.33, open-gap penalty: 2, extended-gap penalty: 2.

A subsequence is a nucleic acid sequence encompassed by SEQ ID NO. 1, SEQ ID NO. 2, SEQ ID NO. 3, SEQ ID NO. 4 and SEQ ID NO. 5, except that one or more nucleotides from the 5′ and/or 3′ end have been deleted and which retains part of or the complete promoter activity. Alternatively, the subsequence may have a higher promoter activity as compared to a nucleic acid sequence selected from the group consisting of SEQ ID NO. 4, SEQ ID NO. 2, SEQ ID NO. 3, SEQ ID NO. 5 and SEQ ID NO. 1. For instance, about 50 or about 100 nucleotides may be deleted from the 5′ and/or 3′ end.

An allelic variant denotes any of two or more alternative forms of a gene occupying the same chromosomal locus. Allelic variation arises naturally through mutation, and may result in polymorphism within populations.

In another embodiment, the present invention relates to isolated nucleic acid sequences which hybridize under conditions of stringency with (i) a sequence selected from the group consisting of SEQ ID NO. 4, SEQ ID NO. 2, SEQ ID NO. 3, SEQ ID NO. 5 and SEQ ID NO. 1; (ii) a subsequence of (i) of at least 100 nucleotides, or (iii) a complementary strand of (i) or (ii).

The subsequence of SEQ ID NO. 1, SEQ ID NO. 2, SEQ ID NO. 3, SEQ ID NO. 4 and SEQ ID NO. 5 may be at least 100 nucleotides or preferably at least 200 nucleotides.

Substantially similar nucleic acid fragments may be characterized by their ability to hybridize to each other under conditions of stringency known by those skilled in the art (Hames and Higgins, Eds. (1985) Nucleic Acid Hybridization, IRL Press, Oxford, UK). Stringency conditions can be adjusted to screen for (highly) similar fragments, such as genes that duplicate functional enzymes from (closely) related organisms. Post hybridization washes determine stringency conditions.

The term “conditions of stringency” is defined herein as a series of washes starting with 6×SSC, 0.5% SDS at room temperature for 15 min, then repeated with 2×SSC, 0.5% SDS at 45° C. for 30 min, and then repeated twice with 0.2×SSC, 0.5% SDS at 60° C. The term “conditions of high stringency” is defined herein as a series of washes starting with 6×SSC, 0.5% SDS at room temperature for 15 min, then repeated with 2×SSC, 0.5% SDS at 45° C. for 30 min, and then repeated twice with 0.1×SSC, 0.1% SDS at 65° C.

For purposes of the present invention, hybridization indicates that the nucleic acid sequence hybridizes to a labeled nucleic acid probe corresponding to the nucleic acid sequence shown in SEQ ID NO. 1, SEQ ID NO. 2, SEQ ID NO. 3, SEQ ID NO. 4 and SEQ ID NO. 5, their complementary strands, or subsequences thereof under conditions of stringency, preferably under conditions of high stringency.

The present invention also relates to a nucleic acid construct comprising a nucleic acid sequence encoding a polypeptide or a nucleic acid, preferably an antisense RNA or a hairpin RNA, operably linked to the nucleic acid sequence described above. It further relates to a recombinant expression vector and a recombinant host cell comprising the nucleic acid construct.

The promoters of the invention show a constitutive, stable, high transcription rate reflecting a high expression of a polypeptide or a nucleic acid, preferably an antisense RNA or a hairpin RNA. With regard to the constitutive and high expression, the more preferred promoters of the invention are stronger, e.g. at least about two to four fold stronger, than e.g. the well known strong actin promoter as seen in Example 6.1.

As a further advantage, the constitutive high expression of the promoters of the invention is stable which is understood to mean that said expression is permanently high over a prolonged period of time, e.g. for several days, e.g. during the fermentative production as herein described. This stable constitutive high expression may be seen e.g. in Examples 1.1, 2.1, 2.1.1., 3.1, 4.1 and 5.1. In contrast to known promoters of which the activity may be influenced e.g. by developmental rhythms and/or by other, e.g. circadian rhythms as discussed above, the high expression of the new promoters of the invention does not seem to be influenced by any such rhythm, but is stable and/or continuously high over the prolonged period of time mentioned above.

In another aspect, the present invention relates to a method for fermentative production of a small organic compound, which small organic compound is obtainable by fermentative production, comprising the steps of:

(a) cultivating a fungal host cell in a medium conductive for the production of a polypeptide or of a nucleic acid, preferably an antisense RNA or a hairpin RNA, which are directed to regulate a metabolic pathway of said host cell thereby leading to the production of said small organic compound, wherein the fungal host cell comprises a first nucleic acid sequence encoding the polypeptide or the nucleic acid, preferably the antisense RNA or the hairpin RNA, operably linked to a second nucleic acid sequence comprising a promoter sequence selected from the group consisting of SEQ ID NO. 4, SEQ ID NO. 2, SEQ ID NO. 3, SEQ ID NO. 5, SEQ ID NO. 1, and subsequences thereof wherein said subsequences are at least 100 nucleotides, and mutant, hybrid, and tandem promoters thereof wherein said mutant has at least about 20% of the promoter activity of said promoter sequences; and
(b) allowing said polypeptide or antisense or hairpin RNA to be expressed; and
(c) isolating the small organic compound from the fermentation broth.

The term “fermentative production” as herein used is understood to mean production by fermentation, e.g. by cultivating a fungal host cell in a nutrient medium and under conditions as herein described.

The polypeptide or the nucleic acid, preferably the antisense RNA or the hairpin RNA directed to regulate a metabolic pathway of said host cell and thereby leading to the production of said small organic compound as described under step (a) may preferably lead to an increase of the production of said small organic compound and/or to a reduction of the production of undesired by-products of said small organic compound.

The small organic compound, e.g. being obtainable by fermentation, as herein described may be selected from—but is in no way limited to—the group of antibiotics, such as β-lactam antibiotics, e.g. a penicillin or a cephalosporin, or HMG-CoA reductase inhibitors such as statins, e.g. lovastatin, or others. Said small organic compound may be obtainable by fermentation of Penicillium chrysogenum, Acremonium chrysogenum and Aspergillus terreus, respectively. The small organic compound may be isolated from the culture medium as described under step (c). Alternatively, said small organic compound may be isolated from the cultivated fungal host cells, e.g. may be extracted from the fungal host cells obtained after the completion of fermentation according to known methods.

Therefore, in another aspect, the present invention relates to the use of a nucleic acid sequence selected from the group consisting of SEQ ID NO. 4, SEQ ID NO. 2, SEQ ID NO. 3, SEQ ID NO. 5, SEQ ID NO. 1 and subsequences thereof, preferably wherein said subsequences are at least 100 nucleotides, for the production of a small organic compound by fermentative production.

In a further aspect, the present invention relates to the use of a nucleic acid sequence, selected from the group consisting of

(a) a nucleic acid sequence having at least 80% homology with a sequence selected from the group consisting of SEQ ID NO. 4, SEQ ID NO. 2, SEQ ID NO. 3, SEQ ID NO. 5 and SEQ ID NO. 1;
(b) a nucleic acid sequence which hybridizes under conditions of stringency with (i) a sequence selected from the group consisting of SEQ ID NO. 4, SEQ ID NO. 2, SEQ ID NO. 3, SEQ ID NO. 5 and SEQ ID NO. 1; (ii) a subsequence of (i) of at least 100 nucleotides, or (iii) a complementary strand of (i) or (ii);
(c) a nucleic acid sequence of (a) or (b) comprising a substitution, deletion, and/or insertion of one or more nucleotides;
(d) an allelic variant of (a), (b), or (c); and
(e) a subsequence of (a), (b), (c), or (d),
wherein the conditions of stringency of (b) are defined as a series of washes starting with 6×SSC, 0.5% SDS at room temperature for 15 min, then repeated with 2×SSC, 0.5% SDS at 45° C. for 30 min, and then repeated twice with 0.2×SSC, 0.5% SDS at 60° C.

The use of the promoters of the invention may lead to an increase in the production titers obtained for the small organic compound and/or may result in a reduction of unwanted by-products of said small organic compounds.

Examples of metabolic pathways which may lead to small organic compounds as herein mentioned and which may be regulated by polypeptides or by nucleic acids, preferably antisense RNAs or hairpin RNAs being themselves under the control of the promoters of the present invention by applying the methods according to the invention, are described below.

The regulation of the genes involved in the biosynthesis, i.e. in the metabolic pathway leading to, penicillin, cephalosporin and/or lovastatin, are under control of a variety of cis-acting DNA-elements and regulatory factors and often tightly linked to developmental regulation. It has been shown for example that over-expression of the laeA gene driven by the strong alcA promoter leads to an up-regulation of genes involved in penicillin and/or lovastatin biosynthesis in Aspergillus terreus and Aspergillus nidulans (Bok, J. W. and Keller, N. P. (2004) Eucaryotic Cell 3(2), 527-535).

Over-expression and/or inactivation of gene expression, either partially or almost completely has shown to be a valuable tool to elucidate novel strategies in order to manipulate key regulators like laeA or metabolic pathways directly or indirectly involved in the biosynthesis and transport of β-lactam antibiotics or lovastatin (Herrmann, M. et al. (2006) Applied and Environmental Microbiology, 72(4), 2957-2970).

Gutierrez and co-workers have shown that over-expression of the cefG gene in Acremonium chrysogenum utilizing promoters of constitutively highly expressed genes leads to increased fermentation titers of cephalosporin and a reduced by-product concentration (Gutierrez S., et al. (1997), Applied Microbiology and Biotechnology 48(5), 606-614).

The promoters of the present invention are e.g. suitable for use in the metabolic pathways described above, e.g. may be used for providing the over-expression of the therein involved genes.

Thus, the promoters of the present invention may be used for manipulating the metabolism, e.g. the metabolic status of an, e.g. filamentous, fungal host cell as herein described.

Furthermore, said promoters may be used to manipulate the growth and/or pathways involved in regulation of morphology and/or sporulation of a fungal host cell, e.g. of a filamentous fungal host as herein described. Thus, the use of the promoters of the invention according to the methods of the invention may lead to a change in the expression of one or more proteins and/or nucleic acids, e.g. RNAs, which are involved in the growth and/or in pathways involved in the regulation of morphology and/or sporulation. Such change in expression may e.g. lead to an over-expression or to a down-regulation of the expression of said proteins or nucleic acids, which may subsequently lead e.g. to improved sporulation of said fungal cell. Additionally, said change in expression of one or more proteins and/or nucleic acids, e.g. RNAs, may lead e.g. to a change of growth, e.g. to improved growth of the fungal host cell. Similarly, the morphology of the fungal host cell may be influenced by applying the promoters of the invention according to the methods of the invention. These manipulations of the growth and/or pathways involved in regulation of morphology and/or sporulation of a fungal host cell may subsequently result in a change in production, e.g. fermentative production, of a metabolite, e.g. of the small organic compound herein described, e.g. may result in an increased fermentative production of said small organic compound, and/or in a reduction of undesired by-products of said small organic compound.

Therefore, the present invention also relates to the use of a nucleic acid sequence of the invention as described above for manipulating the metabolism and/or growth and/or pathways involved in regulation of morphology and/or sporulation of a filamentous fungal host cell.

The fact that the promoters of the invention are constitutive, stable, high expression promoters makes them specifically useful for the methods of producing a polypeptide or a nucleic acid, preferably an antisense RNA or a hairpin RNA, and/or for use in fermentative production of a small organic compound according to the methods of the invention. As a further advantage, the promoters of the invention may be used in the RNA-mediated gene silencing method as herein mentioned.

The following Examples will illustrate the present invention but are not intended to limit the present invention in any way.

The terms “P-Pc X g Y” or “p-Pc X g Y” as used in the examples below are understood to mean the “Pc X g Y promoter” which itself is synonymous to the terms “Pc X g Y promoter region”. E.g. the terms “P-Pc12g09320” used in example 1.3 are synonymous to, i.e. mean the “Pc12g09320 promoter”—as mentioned e.g. in example 1.4—and are synonymous to, i.e. mean the “Pc12g09320 promoter region”—as mentioned e.g. in example 1.2.

### EXAMPLES

### Example 1

### 1.1. Quantitative PCR Expression Analysis of the Pc12g09320 Gene of P. chrysogenum

In order to confirm the expression pattern and to quantify the high expression level of the new gene Pc12g09320 quantitative PCR analysis is performed (ABI7900 HT, Applied Biosystems). Total RNA from mycelia of P. chrysogenum is isolated which are grown under various conditions, i.e. under production conditions and collected by filtration on sterile cloth. The wet mycelium is flash-frozen in liquid nitrogen and ground to a powder using a mortar and pestle. Total RNA is extracted using the TRIZOL® Reagent (Invitrogen) extraction protocol. All procedures are performed according to the manufacturers protocols. After extraction the RNA samples are purified with RNeasy® columns (Qiagen) and an on-column DNase digest is performed using the RNase-Free DNase Set Kit (Qiagen). RNA is re-suspended in DEPC-treated, sterile, destilled water and its concentration is measured by spectrophotometry (Ultrospec 3100 pro, Amersham). The quality of the RNA is checked by Bioanalyzer-measurements (Agilent) using the RNA 6000 Nano Assay (Agilent). Subsequent reverse transcription is performed using the High Capacity cDNA Archive Kit (Applied Biosystems). Specific oligonucleotides for the Pc12g09320 gene are designed using the Primer Express software (Applied Biosystems). The forward primer Pc12g09320_U1 (5′-CAAGGCCGACCACAGCTT-3′) (SEQ ID NO. 6) and the reverse primer Pc12g09320_L1 (5′-GCCGAGACGGTTGACGAAT-3′) (SEQ ID NO. 7) which cover positions 207-224 and 306-288, respectively, of the Pc12g09320 gene sequence are designed. As a control, the known act gene of P. chrysogenum is used (Acc. No. AF056975) for the design of the forward primer Pc_actA_U1 (5′-GGTGATGAGGCACAGTCGAAG-3′) (SEQ ID NO. 8) and the reverse primer Pc_actA_L1 (5′-AGCTCGTTGTAGAAGGTGTGGTG-3′) (SEQ ID NO. 9) which yield a 119-bp amplicon. For PCR reactions the SYBR Green Mastermix (Applied Biosystems) is used. The reaction is performed according to the manufacturers protocol using 20 μl reactions which each contained 50 ng of template cDNA. A no template control (NTC), with no added template RNA to control for any contaminants in reagents for each template is included. The good efficiency of all primer sets used is validated by standard curves. The results confirm the very strong and constitutive gene expression levels which are also detected by full genome microarray analysis. In particular this permanent and constitutive expression level is detected under production conditions.

### 1.2. Cloning and Characterization of the Pc12g09320 Promoter Region

To create a suitable recipient reporter gene vector the plasmid pGen4.5 is generated in two steps. In the first step a 607 bp terminator region of the Penicillium chrysogenum γ-actin gene (Accession No. AF056975) is amplified by PCR with the proof reading, Pwo DNA polymerase (Roche), digested with NotI and BamHI and cloned in a pBluescript II KS+Vector from Stratagene. In the next step a 1812 bp fragment including the coding region of the E. coli uidA gene is amplified by PCR with the proof reading, Pwo DNA polymerase (Roche), digested with NarI and PstI and cloned in the pBluescript II KS+Vector containing the γ-actin terminator region.

On the basis of the Penicillium chrysogenum genome the putative promoter region of the gene Pc12g09320 is evaluated assuming that the coding region of the gene Pc12g09310 upstream of Pc12g09320 delimits its putative promoter region.

The Pc 2g09320 promotor region is cloned using the following oligonucleotides as primers:

Pc12g09320-for

(SEQ ID NO. 10)

(5′-GTCAAAGCTTGTGTACTTACCAATGGC-3′)
and
Pc12g09320-rev

(SEQ ID NO. 11)

(5′-AGTCATGCATGCTGAATGAAGGCGGGAGA-3′).

The 351 bp DNA fragment obtained by PCR with the proof reading, Pwo DNA polymerase (Roche) is digested with HindIII and SphI and subcloned in the HindIII and SphI digested plasmid pGen4.5 containing the E. coli uidA gene and the Penicillium chrysogenum act terminator region and generating the plasmid pGen4.24. The uidA gene of E. coli is fused translationally with the Pc 2g09320 promotor region. This is carried out designing the SphI site such that it is part of the ATG start codon which codes for the initiator methionine of the uidA gene.

The sequencing of the amplified promoter region as well as the fusion region between p-Pc12g09320 and the uidA gene confirm the correctness of the promoter sequence as well as the exact arrangement of the latter reporter gene in the correct reading frame.

### 1.3. Expression of the uidA Gene of E. coli in P. chrysogenum Driven by the P-Pc12g09320

For cotransformation of pGen4.24 into P. chrysogenum strain Q176 the plasmid pBC1003 carrying the Tn5-phleomycin resistance gene under the control of the isopenicillin-N-synthetase promoter from P. chrysogenum is used as the selection marker (2). Protoplasts are transformed according to Cantoral et al. (1), and transformants are selected on minimal medium containing NaNO₃ and glucose as the nitrogen and carbon sources, respectively and 30 μg/ml phleomycin (Sigma). Screening of positive clones is performed by UIDA-Plate assay on minimal medium containing NaNO₃ and glucose as the nitrogen and carbon sources and 50 μg/ml of the cyclohexylammonium salt of 5-bromo-4-chloro-3-indolyl-β-D-glucuronic acid (Biosynth) as substrat for the UIDA protein. Positive clones can be identified through their ability to generate blue coloured colonies after 2-5 days incubation at 25° C. To obtain homokaryotic transformants, colonies from single homokaryotic spores are picked, and genomic integration of the expression construct is verified by Southern analysis. Four independent transformants transformants carrying a copy of pGen 4.24 integrated ectopically are selected for growth tests, Northern blot and qPCR analysis.

Southern analysis confirms the presence of the plasmid pGen4.24. The presence of the UIDA transcript can be determined by Northern Blot analysis and the relative amount by qPCR-analysis.

UIDA-Plate assay as well as Northern and qPCR analysis show clearly that the transformed P-Pc12g09320-uidA construct expresses the heterologous bacterial &glucuronidase gene.

### 1.4. Expression of the uidA Gene in P. chrysogenum Under Control of the Pc12g09320 Promoter

The uidA gene of E. coli is fused with the Pc 2g09320 promoter as described in Section 1.2. of Example 1. The heterologous expression of the uidA gene is characterized by quantitative PCR (7900HT, Applied Biosystems). Specific oligonucleotides for the uidA gene are designed using the Primer Express software (Applied Biosystems). The forward primer Ec_uidA_U1 (5′-TTCATGCCAGTCCAGCGTT-3′) (SEQ ID NO. 12) and the reverse primer Ec_uidA_L1 (5′-CGACCGCAAACCGAAGTC-3′) (SEQ ID NO. 13) which yield a 56-bp product. The processes of RNA isolation and purification as well as the cDNA synthesis and the subsequents quantitative PCR analysis using the act gene as control are carried out as described in Section 1.1. of Example 1. Four independent P. chrysogenum strains which harbor the uidA gene under control of the Pc12g09320 promoter are analyzed by qPCR. In all of these strains a very high level of uidA mRNA is detected. This result confirms that the Pc12g09320-promoter-uidA construct expresses the heterologous uidA gene as well as the endogenous Pc12g09320 gene. This result is in accordance with the strong UIDA enzyme activity which is observed on chromogenic plate medium (see Section 1.3., Example 1).

### Example 2

### 2.1. Quantitative PCR expression analysis of the Pc12g10000 gene of P. chrysogenum

Quantitative PCR analysis is performed to confirm and quantify the high expression level of new gene Pc12g09320 which is detected by full genome microarray analysis. qPCR analysis is performed as described in Section 1.1. of Example 1. Total RNA is isolated from mycelia of P. chrysogenum which are grown under various conditions, i.e. under production conditions. Subsequent reverse transcription is performed to generate cDNA which serves as template in qPCR analysis (see Section 1.1. of Example 1). Specific oligonucleotides for the Pc12g10000 gene are designed using the Primer Express software (Applied Biosystems). The forward primer Pc12g10000U1 (5′-AACMCATTGCGCTCGATGA-3′) (SEQ ID NO. 14) and the reverse primer Pc12g10000_L1 (5′-CCTTGGAAACCGTGTTCGTT-3′) (SEQ ID NO. 15) which cover positions 100-119 and 199-180, respectively, of the Pc12g10000 gene sequence are designed. The results confirm the very strong and constitutive gene expression levels which are also detected by full genome microarray analysis. The analysis confirms the permanent and constitutive expression level of Pc12g09320 under production conditions.

### 2.1.1 Quantitative PCR Expression Analysis of the Pc12g10000 Gene of P. chrysogenum

Quantitative PCR analysis is performed to confirm and quantify the high expression level of the new gene Pc12g10000 which is detected by full genome microarray analysis. qPCR analysis is performed as described in Section 1.1. of Example 1. Total RNA is isolated from mycelia of P. chrysogenum which are grown under various conditions, i.e. under production conditions. Subsequent reverse transcription is performed to generate cDNA which serves as template in qPCR analysis (see Section 1.1. of Example 1). Specific oligonucleotides for the Pc12g10000 gene are designed using the Primer Express software (Applied Biosystems). The forward primer Pc12g10000_U1 (5′-AACMCATTGCGCTCGATGA-3′) (SEQ ID NO. 14) and the reverse primer Pc12g10000_L1 (5′-CCTTGGAAACCGTGTTCGTT-3′) (SEQ ID NO. 15) which cover positions 100-119 and 199-180, respectively, of the Pc12g10000 gene sequence are designed. The results confirm the very strong and constitutive gene expression levels which are also detected by full genome microarray analysis. The analysis confirms the permanent and constitutive expression level of Pc12g10000 under production conditions.

### 2.2. Cloning and Characterization of the Pc12g10000 Promoter Region

The Pc12g10000 promotor region is cloned using the following oligonucleotides as primers:

Pc12g10000-for

(SEQ ID NO. 16)

(5′-GTCAAAGCTTCCGGCTCGGATCTCGTC-3′)
and
Pc12g10000-rev

(SEQ ID NO. 17)

(5′-GAGGGCATGCTGACTTGTTCACTTCAAGG-3′).

The 764 bp DNA fragment obtained by PCR with the proof reading, Pwo DNA polymerase (Roche) is digested with HindIII and SphI and subcloned in the HindIII and SphI digested plasmid pGen4.5 containing the E. coli uidA gene and the Penicillium chrysogenum act terminator region and generating the plasmid pGen4.25 (see section 1.2., Example 1). The uidA gene of E. coli is fused translationally with the Pc12g10000 promotor region. This is carried out designing the SphI site such that it is part of the ATG start codon which codes for the initiator methionine of the uidA gene.

The sequencing of the amplified promoter region as well as the fusion region between p-Pc12g10000 and the uidA gene confirm the correctness of the promoter sequence as well as the exact arrangement of the latter reporter gene in the correct reading frame.

### 2.3. Expression of the uidA Gene of E. coli in P. chrysogenum Driven by the P-Pc12g10000

pGen4.25 is transformed and the transformation event verified by the same procedure as described in section 1.3., Example 1.

Southern analysis confirms the presence of the plasmid pGen4.25. The presence of the UIDA transcript can be determined by Northern Blot analysis and the relative amount by qPCR-analysis. UIDA-Plate assay as well as Northern and qPCR analysis show clearly that the transformed P-Pc12g10000-uidA construct expresses the heterologous bacterial β-glucuronidase gene.

### 2.4. Expression of the uidA Gene in P. chrysogenum Under Control of the Pc12g10000 Promoter

The uidA gene of E. coli is fused with the Pc12g09320 promoter as described in Section 2.2. of Example 2. The heterologous expression of the uidA gene is characterized by quantitative PCR (7900 HT, Applied Biosystems). Specific oligonucleotides for the uidA gene are designed as described in Section 1.4. of Example 1. The processes of RNA isolation and purification as well as the cDNA synthesis and the subsequents quantitative PCR analysis using the act gene as control are carried out as described in Section 1.1. of Example 1. Three independent P. chrysogenum strains which harbor the uidA gene under control of the Pc12g10000 promoter are analyzed by qPCR. In all of these strains a very high level of uidA mRNA is detected. This result confirms that the 12g10000-promoter-uidA construct expresses the heterologous uidA gene as well as the endogenous Pc12g09320 gene. This result is in accordance with the strong UIDA enzyme activity which is observed on chromogenic plate medium (see Section 2.3., Example 2).

### 2.4.1 Expression of the uidA Gene in P. chrysogenum Under Control of the Pc12g10000 Promoter

The uidA gene of E. coli is fused with the Pc12g10000 promoter as described in Section 2.2. of Example 2. The heterologous expression of the uidA gene is characterized by quantitative PCR (7900HT, Applied Biosystems). Specific oligonucleotides for the uidA gene are designed as described in Section 1.4. of Example 1. The processes of RNA isolation and purification as well as the cDNA synthesis and the subsequents quantitative PCR analysis using the act gene as control are carried out as described in Section 1.1. of Example 1. Three independent P. chrysogenum strains which harbor the uidA gene under control of the Pc12g10000 promoter are analyzed by qPCR. In all of these strains a very high level of uidA mRNA is detected. This result confirms that the Pc12g10000-promoter-uidA construct expresses the heterologous uidA gene as well as the endogenous Pc12g10000 gene. This result is in accordance with the strong UIDA enzyme activity which is observed on chromogenic plate medium (see Section 2.3., Example 2).

### Example 3

### 3.1. Quantitative PCR Expression Analysis of the Pc16g00660 Gene of P. chrysogenum

Quantitative PCR analysis is performed to confirm and quantify the high expression level of the new gene Pc16g00660 which is detected by full genome microarray analysis. qPCR analysis is performed as described in Section 1.1. of Example 1. Total RNA is isolated from mycelia of P. chrysogenum which are grown under various conditions, i.e. under production conditions. Subsequent reverse transcription is performed to generate cDNA which served as template in qPCR analysis (see Section 1.1. of Example 1). Specific oligonucleotides for the Pc16g00660 gene are designed using the Primer Express software (Applied Biosystems). The forward primer Pc16g00660_U1 (5′-TGAAGTTCGACGAGGACTGATG-3′) (SEQ ID NO. 18) and the reverse primer Pc16g00660_L1 (5′-CGCTTGAGACGTCAGGTTGTT-3′) (SEQ ID NO. 19) which cover positions 1122-1143 and 1221-1201, respectively, of the Pc16g00660 gene sequence are designed. The qPCR analysis confirms the very strong and constitutive gene expression levels which are also detected by full genome microarray analysis. The analysis confirms the permanent and constitutive expression level of Pc16g00660 under production conditions.

### 3.2. Cloning and Characterization of the Pc16g00660 Promoter Region

The Pc16g00660 promotor region is cloned using the following oligonucleotides as primers:

Pc16g00660-for

(SEQ ID NO. 20)

(5′-GTCAAAGCTTGATATGTGGAGCCTGCG-3′)
and
Pc16g00660-rev

(SEQ ID NO. 21)

(5′-TCATGCATGCTGGCGGTTCTGGAATCCAG-3′).

The 1498 bp DNA fragment obtained by PCR with the proof reading, Pwo DNA polymerase (Roche) is digested with HindIII and SphI and subcloned in the HindIII and SphI digested plasmid pGen4.5 containing the E. coli uidA gene and the Penicillium chrysogenum act terminator region and generating the plasmid pGen4.26 (see section 1.2., Example 1). The uidA gene of E. coli is fused translationally with the Pc16g00660 promotor region. This is carried out designing the SphI site such that it is part of the ATG start codon which codes for the initiator methionine of the uidA gene.

The sequencing of the amplified promoter region as well as the fusion region between p-Pc16g00660 and the uidA gene confirm the correctness of the promoter sequence as well as the exact arrangement of the latter reporter gene in the correct reading frame.

### 3.3. Expression of the uidA Gene of E. coli in P. chrysogenum Driven by the P-Pc16g00660

pGen4.26 is transformed and the transformation event verified by the same procedure as described in section 1.3., Example 1.

Southern analysis confirms the presence of the plasmid pGen4.26. The presence of the UIDA transcript can be determined by Northern Blot analysis and the relative amount by qPCR-analysis. UIDA-Plate assay as well as Northern and qPCR analysis show clearly that the transformed P-Pc16g00660-uidA construct expresses the heterologous bacterial β-glucuronidase gene.

### 3.4. Expression of the uidA Gene in P. chrysogenum Under Control of the Pc16g00660 Promoter

The uidA gene of E. coli is fused with the Pc16g00660 promoter as described in Section 3.2. of Example 3. The heterologous expression of the uidA gene is characterized by quantitative PCR (7900HT, Applied Biosystems). Specific oligonucleotides for the uidA gene are designed as described in Section 1.4. of Example 1. The processes of RNA isolation and purification as well as the cDNA synthesis and the subsequent quantitative PCR analysis using the act gene as control are carried out as described in Section 1.1. of Example 1. Four independent P. chrysogenum strains which harbor the uidA gene under control of the Pc16g00660 promoter are analyzed by qPCR. In all of these strains a very high level of uidA mRNA is detected. This result confirms that the Pc16g00660-promoter-uidA construct expresses the heterologous uidA gene as well as the endogenous Pc16g00660 gene. This result is in accordance with the strong UIDA enzyme activity which is observed on chromogenic plate medium (see Section 3.3., Example 3).

### Example 4

### 4.1. Quantitative PCR Expression Analysis of the Pc21g04830 Gene of P. Chrysogenum

Quantitative PCR analysis is performed to confirm and quantify the high expression level of the new gene Pc21 g04830 which is detected by full genome microarray analysis. qPCR analysis is performed as described in Section 1.1. of Example 1. Total RNA is isolated from mycelia of P. chrysogenum which are grown under various conditions, i.e. under production conditions. Subsequent reverse transcription is performed to generate cDNA which served as template in qPCR analysis (see Section 1.1 of Example 1). Specific oligonucleotides for the Pc21g04830 gene are designed using the Primer Express software (Applied Biosystems). The forward primer Pc21g04830_U1 (5′-TTGTCCTGGTCTTTCCCCATT-3′) (SEQ ID NO. 22) and the reverse primer Pc21g04830_L1 (5′-CCAGGCCTACCGTACCATTG-3′) (SEQ ID NO. 23) which cover positions 490-510 and 589-570, respectively, of the Pc21g04830 gene sequence are designed. The qPCR analysis confirmed the very strong and constitutive gene expression levels which are also detected by full genome microarray analysis. The analysis confirms the permanent and constitutive expression level of Pc21g04830 under production conditions.

### 4.2. Cloning and Characterization of the Pc21g04830 Promoter Region

The Pc21g04830 promoter region is cloned using the following oligonucleotides as primers:

Pc21g04830-for

(SEQ ID NO. 24)

(5′-GTCAAAGCTTGAATTCCATCGCCGGGTCGCC-3′)
and
Pc21g04830-rev

(SEQ ID NO. 25)

(5′-TCATGCATGCTCGAGGAAGGGAGGAGAGG-3′).

The 1363 bp DNA fragment obtained by PCR with the proof reading, Pwo DNA polymerase (Roche) is digested with HindIII and SphI and subcloned in the HindIII and SphI digested plasmid pGen4.5 containing the E. coli uidA gene and the Penicillium chrysogenum act terminator region and generating the plasmid pGen4.27 (see section 1.2., Example 1). The uidA gene of E. coli is fused translationally with the Pc21g04830 promotor region. This is carried out designing the SphI site such that it is part of the ATG start codon which codes for the initiator methionine of the uidA gene.

The sequencing of the amplified promoter region as well as the fusion region between p-Pc21g04830 and the uidA gene confirm the correctness of the promoter sequence as well as the exact arrangement of the latter reporter gene in the correct reading frame.

### 4.3. Expression of the uidA Gene of E. coli in P. chrysogenum Driven by the P-Pc21g04830

pGen4.27 is transformed and the transformation event verified by the same procedure as described in section 1.3., Example 1.

Southern analysis confirms the presence of the plasmid pGen4.27. The presence of the UIDA transcript can be determined by Northern Blot analysis and the relative amount by qPCR-analysis. UIDA-Plate assay as well as Northern and qPCR analysis show clearly that the transformed P-Pc21g04830-uidA construct expresses the heterologous bacterial β-glucuronidase gene.

### 4.4. Expression of the uidA Gene in P. chrysogenum Under Control of the Pc21g04830 Promoter

The uidA gene of E. coli is fused with the Pc21g04830 promoter as described in Section 4.2. of Example 4. The heterologous expression of the uidA gene is characterized by quantitative PCR (7900HT, Applied Biosystems). Specific oligonucleotides for the uidA gene are designed as described in Section 1.4. of Example 1. The processes of RNA isolation and purification as well as the cDNA synthesis and the subsequent quantitative PCR analysis using the act gene as control are carried out as described in Section 1.1. of Example 1. Four independent P. chrysogenum strains which harbor the uidA gene under control of the Pc21g04830 promoter are analyzed by qPCR. In all of these strains a very high level of uidA mRNA is detected. This result confirms that the Pc21g04830-promoter-uidA construct expresses the heterologous uidA gene as well as the endogenous Pc 6g00660 gene. This result is in accordance with the strong UIDA enzyme activity which is observed on chromogenic plate medium (see Section 3.3., Example 3).

### Example 5

### 5.1. Quantitative PCR Expression Analysis of the Pc21g20300 Gene of P. chrysogenum

Quantitative PCR analysis is performed to confirm and quantify the high expression level of the new gene Pc21g20300 which is detected by full genome microarray analysis. qPCR analysis is performed as described in Section 1.1. of Example 1. Total RNA is isolated from mycelia of P. chrysogenum which are grown under various conditions, i.e. under production conditions. Subsequent reverse transcription is performed to generate cDNA which served as template in qPCR analysis (see Section 1.1. of Example 1). Specific oligonucleotides for the Pc21g20300 gene are designed using the Primer Express software (Applied Biosystems). The forward primer Pc21g20300_U1 (5′-CTGCAGAGCGATGGTTGCT-3′) (SEQ ID NO. 26) and the reverse primer Pc21g20300_L1 (5′-TTGGAGACAGAGACTTGGTCCTT-3′) (SEQ ID NO. 27) which cover positions 103-121 and 230-208, respectively, of the Pc21g20300 gene sequence are designed. The qPCR analysis confirms the very strong and constitutive gene expression levels which are also detected by full genome microarray analysis. The analysis confirms the permanent and constitutive expression level of Pc21g20300 under production conditions.

### 5.2. Cloning and Characterization of the Pc21g20300 Promoter Region

The Pc21g20300 promoter region is cloned using the following oliqonucleotides as primers:

| | |
|---|---|
| Pc21g20300-for | |
| (5′-GTCAGAATTCCCCTTCTGGTGATTG-3′) | (SEQ ID NO. 28) |
| and | |
| Pc21g20300-rev | |
| (5′-GCTGATGCATCTTGATGGATTGACT-3′). | (SEQ ID NO. 29) |

The 1359 bp DNA fragment obtained by PCR with the proof reading, Pwo DNA polymerase (Roche) is digested with EcoRI and NsiI and subcloned in the EcoRI and NsiI digested plasmid pGen4.5 containing the E. coli uidA gene and the Penicillium chrysogenum act terminator region and generating the plasmid pGen4.30 (see section 1.2., Example 1). The uidA gene of E. coli is fused translationally with the Pc21g20300 promotor region. This is carried out designing the NsiI site such that it is part of the ATG start codon which codes for the initiator methionine of the uidA gene.

The sequencing of the amplified promoter region as well as the fusion region between p-Pc21g20300 and the uidA gene confirm the correctness of the promoter sequence as well as the exact arrangement of the latter reporter gene in the correct reading frame.

### 5.3. Expression of the uidA Gene of E. coli in P. chrysogenum Driven by the P-Pc21 g20300

pGen4.30 is transformed and the transformation event verified with the same procedure described in section 1.3., Example 1.

Southern analysis confirms the presence of the plasmid pGen4.30. The presence of the UIDA transcript can be determined by Northern Blot analysis and the relative amount by qPCR-analysis. UIDA-Plate assay as well as Northern and qPCR analysis show clearly that the transformed P-Pc21g20300-uidA construct expresses the heterologous bacterial β-glucuronidase gene.

### 5.4. Expression of the uidA Gene in P. chrysogenum Under Control of the Pc21g20300 Promoter

The uidA gene of E. coli is fused with the Pc21g20300 promoter as described in Section 5.2. of Example 5. The heterologous expression of the uidA gene is characterized by quantitative PCR (7900HT, Applied Biosystems). Specific oligonucleotides for the uidA gene are designed as described in Section 1.4. of Example 1. The processes of RNA isolation and purification as well as the cDNA synthesis and the subsequent quantitative PCR analysis using the act gene as control are carried out as described in Section 1.1. of Example 1. Three independent P. chrysogenum strains which harbor the uidA gene under control of the Pc21g20300 promoter are analyzed by qPCR. In all of these strains a very high level of uidA mRNA is detected. This result confirms that the Pc21g20300-promoter-uidA construct expresses the heterologous uidA gene as well as the endogenous Pc21g20300 gene. This result is in accordance with the strong UIDA enzyme activity which is observed on chromogenic plate medium (see Section 5.3, Example 3).

### Example 6

### Comparison of the New Promoters P-Pc21g04830, P-Pc12g 0000 and P-Pc16g00660 Expression Strength to the Known Actin Promoter of Penicillium chrysogenum by Quantitative PCR Expression Analysis

Comparative quantitative PCR analysis is performed to compare the quantity of the expression of the reporter gene uidA which was fused to the new promoters P-Pc21g04830 (SEQ ID NO. 4), P-Pc12g10000 (SEQ ID NO. 2) and P-Pc16g00660 (SEQ ID NO. 3) to the expression level of the well known Penicillium chrysogenum actin promoter (Gen Bank Accession No. AF056975), which is also fused to uidA, e.g. as described in U.S. Pat. No. 6,300,095 B1. The fusion of the new promoters P-Pc21g04830, P-Pc12g1000 and P-Pc16g00660 to the reporter gene uidA is performed as described in section 4.4 of Example 4, Section 2.4.1 of Example 2 and Section 3.4 of Example 3, respectively. For comparison of the expression levels the Penicillium chrysogenum actin promoter region (U.S. Pat. No. 6,300,095 B1, Gen Bank Accession No. AF056975) is translationally fused to the uidA gene of E. coli. The actin promoter region is cloned using the following oligonucleotides as primers:

acnP-P5B

(SEQ ID NO. 30)

(5′- CACTTAAGCTTCCCGTATTATCCCCATC -3′)
and
acnP-P3B

(SEQ ID NO. 31)

(5′- CCCTATGCATGCGTGACTGATTAAACAAGGG -3′).

The 648 bp DNA fragment obtained by PCR with the proof reading, Pwo DNA polymerase (Roche) is digested with HindIII and SphI and subcloned in the HindIII and SphI digested plasmid pGen4.5 containing the E. coli uidA gene and the Penicillium chrysogenum act terminator region and generating the plasmid pGen4.3. The uidA gene of E. coli is fused translationally with the actin promotor region. This is carried out designing the SphI site such that it is part of the ATG start codon which codes for the initiator methionine of the uidA gene.

The sequencing of the amplified promoter region as well as the fusion region between the actin promotor and the uidA gene confirm the correctness of the promoter sequence as well as the exact arrangement of the latter reporter gene in the correct reading frame.

The heterologous expression of the uidA gene is analyzed by quantitative PCR (7900HT, Applied Biosystems). Therefore specific oligonucleotides for the uidA gene are designed as described in Section 1.4. of Example 1. The processes of RNA isolation and purification as well as the cDNA synthesis and the subsequent quantitative PCR analysis using the actin gene as control are carried out as described in Section 1.1. of Example 1.

The results confirm that the Pc21g04830-promoter-uidA construct as well as the, Pc12g10000-promoter-uidA construct as well as the Pc16g00660-promoter-uidA construct express the uidA gene at least two to four fold stronger than the Penicillium chrysogenum actin-promoter-uidA construct. The data clearly prove P-Pc21g04830, P-Pc12g10000 and P-Pc16g00660 being much stronger promoters than the known actin promoter of Penicillium chrysogenum.

### CITED REFERENCES

1 Cantoral, I. et al. “High frequency transformation of Penicillium chrysogenum” J. Biotechnol. 5 (1987) pp. 494-497.
2 Kolar, M. et al. “Transformation of Penicillium chrysogenum using dominant selection markers and expression of an Escherichia coli lacZ fusion gene” Gene 62 (1) (1988) pp. 127-134.
3 Zadra, I. et al. “xylP promoter-based expression system and its use for antisense downregulation of the Penicillium chrysogenum nitrogen regulator NRE” Appl. Environ. Microbiol. 66(11) (2000) pp. 4810-4816.
4 Bautista, L. F. et al. “Antisense Silencing of the creA Gene in Aspergillus nidulans” Appl. Environ. Microbiol. 66(10) (2000) pp. 4579-4581.
5 Wang, T. H. et al. “Antisense inhibition of xylitol dehydrogenase gene, xdh1 from Trichoderma reesei”
   Lett. Appl. Microbiol. 40 (2005) pp. 424-429.
6 Nakayashiki, H. et al. “RNA silencing as a tool for exploring gene function in ascomycete fungi”
   Fungal Genetics and Biology 42 (2005) pp. 275-283.
7 Kadotani, N., et al. “RNA-silencing in the Phytopathogenic Fungus Magnaporthe oryzae”
   Mol. Plant-Microbiol. Interactions 16(9) (2003) pp. 769-776.
8 Mouyna, I., et al. “Gene silencing with RNA interference in the human pathogenic fungus Aspergillus fumigatus”
   FEMS Microbiol. Lett. 237 (2004) pp. 317-324.
9 Finklstein, Ball (eds.), Applied Molecular Genetics of Filamentous fungi (ibid.) Biotechnology of Filamentous fungi, Butterworth-Heinemann, Boston (1992).
10 Hames and Higgins (eds.) Nucleic Acid Hybridization, IRL Press, Oxford, UK (1985).
11 Bok, J. W. and Keller, N. P. “LaeA, a regulator of Secondary Metabolism in Aspergillus spp”
   Eucaryotic Cell 3(2) (2004) pp. 527-535.
12 Herrmann M. et al. “Protein Kinase C (PkcA) of Aspergillus nidulans is Involved In Penicillin Production”
   Applied and Enviromental Microbiology 72 (4) (2006) pp. 2957-2970.
13 Gutierrez S., et al. “Expression of the cefG gene is limiting for cephalosporin biosynthesis in Acremonium chrysogenum”
   Applied Microbiology and Biotechnology 48(5) (1997) pp. 606-614.
14. Cooper G. M., “The Cell. A Molecular Approach”, 2^{nd} edition (2000) online version.
15. Alberts et al., “Molecular Biology of the Cell”, 4^{th} Edition (2002) online version.
16. Jeong, H Y. et al. “Differential expression of house-keeping genes of Aspergillus nidulans during sexual development”
   Gene 262 (2001) pp. 215-219.
17. Greene A. V., et al. “A circadian oscillator in Aspergillus spp. regulates daily development and gene expression”
   Eukaryotic Cell 2 (2) (2003) pp. 231-237.
18. Gutierrez, S. et al. “Transcription of the pcbAB, pcbC and penDE genes of Penicillium chrysogenum AS-P-78 is repressed by glucose and the repression is not reversed by alkaline pHs”
   Microbiology 145 (1999) pp. 317-324.

1. A method for producing a polypeptide or a nucleic acid, preferably an antisense RNA or a hairpin RNA, comprising the steps of:
(a) cultivating a fungal host cell in a medium conductive for the production of the polypeptide or the nucleic acid, preferably the antisense RNA or the hairpin RNA, wherein the fungal host cell comprises a first nucleic acid sequence encoding the polypeptide or the nucleic acid, preferably the antisense RNA or the hairpin RNA, operably linked to a second nucleic acid sequence comprising a promoter sequence selected from the group consisting of SEQ ID NO. 4, SEQ ID NO. 2, SEQ ID NO. 3, SEQ ID NO. 5, SEQ ID NO. 1, and subsequences thereof wherein said subsequences are at least 100 nucleotides, and mutant, hybrids and tandem promoters thereof wherein said mutant has at least about 20% of the promoter activity of said promoter sequences; and
(b) isolating the polypeptide from the culture medium, or
(c) isolating the polypeptide from the fungal host cell.

2. A method according to claim 1, wherein the fungal host cell in step (a) and/or in step (c) is a filamentous fungal host cell.

3. A method according to claim 1, wherein the fungal host cell in step (a) and/or in step (c) is Penicillium chrysogenum or Acremonium chrysogenum or Aspergillus terreus or Penicillium citrinum.

4. Isolated nucleic acid sequence comprising a nucleic acid sequence selected from the group consisting of SEQ ID NO. 4, SEQ ID NO. 2, SEQ ID NO. 3, SEQ ID NO. 5, SEQ ID NO. 1 and subsequences thereof wherein said subsequences are at least 100 nucleotides.

5. Isolated nucleic acid sequence, selected from the group consisting of
(a) a nucleic acid sequence having at least 80% homology with a sequence selected from the group consisting of SEQ ID NO. 4, SEQ ID NO. 2, SEQ ID NO. 3, SEQ ID NO. 5 and SEQ ID NO. 1;
(b) a nucleic acid sequence which hybridizes under conditions of stringency with (i) a sequence selected from the group consisting of SEQ ID NO. 4, SEQ ID NO. 2, SEQ ID NO. 3, SEQ ID NO. 5 and SEQ ID NO. 1; (ii) a subsequence of (i) of at least 100 nucleotides, or (iii) a complementary strand of (i) or (ii);
(c) a nucleic acid sequence of (a) or (b) comprising a substitution, deletion, and/or insertion of one or more nucleotides;
(d) an allelic variant of (a), (b), or (c); and
(e) a subsequence of (a), (b), (c), or (d),
wherein the conditions of stringency of (b) are defined as a series of washes starting with 6×SSC, 0.5% SDS at room temperature for 15 min, then repeated with 2×SSC, 0.5% SDS at 45° C. for 30 min. and then repeated twice with 0.2×SSC, 0.5% SDS at 60° C.

6. A nucleic acid construct comprising a nucleic acid sequence encoding a polypeptide or a nucleic acid, preferably an antisense RNA or a hairpin RNA, operably linked to the nucleic acid sequence of claim 4.

7. A recombinant expression vector comprising the nucleic acid construct of claim 6.

8. A recombinant host cell comprising the nucleic acid construct of claim 6.

9. A method for fermentative production of a small organic compound, which small organic compound is obtainable by fermentative production, comprising the steps of:
(a) cultivating a fungal host cell in a medium conductive for the production of a polypeptide or of a nucleic acid, preferably an antisense RNA or a hairpin RNA, which are directed to regulate a metabolic pathway of said host cell thereby leading to the production of said small organic compound, wherein the fungal host cell comprises a first nucleic acid sequence encoding the polypeptide or the nucleic acid, preferably the antisense RNA or the hairpin RNA, operably linked to a second nucleic acid sequence comprising a promoter sequence selected from the group consisting of SEQ ID NO. 4, SEQ ID NO. 2, SEQ ID NO. 3, SEQ ID NO. 5, SEQ ID NO. 1, and subsequences thereof wherein said subsequences are at least 100 nucleotides, and mutant, hybrid, and tandem promoters thereof wherein said mutant has at least about 20% of the promoter activity of said promoter sequences; and
(b) allowing said polypeptide or antisense or hairpin RNA to be expressed; and
(c) isolating the small organic compound from the fermentation broth.

10. Use of a nucleic acid sequence according to 4 for the production of a small organic compound by fermentative production.

11. Use of a nucleic acid sequence according to 4 for manipulating the metabolism and/or growth and/or pathways involved in regulation of morphology and/or sporulation of a filamentous fungal host cell.


SEQUENCE LISTING (31 sequences)

SEQ ID NO: 1 - DNA, 331 bp, Penicillium chrysogenum
ttgtgtacttaccaatggctatctctcataaactatcgaagcctggtcctttcttaaagccagccacataaagctacctaggtagcttcagcccgtgatcaaacccaatgctaccctgcaacatacactattttgactattgacttatctcttgtaatctggacttggtcttgcctactctgggggtacatcgtacaaacttgctatcttttactttttttgcttttaagatctcaatgtctcctttttaatcttccttctcactcacacacctcttaatattcccacaccttttatcgtctattcttctgcggtctcccgccttcattca

SEQ ID NO: 2 - DNA, 744 bp, Penicillium chrysogenum
ccggctcggatctcgtcatagagcaaggggcattttgatacggatttgcggctcttggagatagttggtaagatatcatcggtgacattgtgtttatatctcactcctttcatcgttaaagaacacactgtaattgatattgtggtgatcgagagtacttgaaatataaagacatttttctgaacaggtgattataaatatgcgcaacaacaccggggactgtcgaccatcgaatatcatattcaaattttgcggctcaaaccaaaaaagaatatactgtatgttctaaggttgtagtctatcctccgcagaaccagaactacgagctacgctgccccaccttgtccgaatgtagcatagtaaccatgcagatccttgccaaatccttcatccctgcagccccctgaccagatacctaagcaggtaaccttagaagacaagagaaccagtgattacaaatccgacttcggtgtaagcattggaatttcttttgctgaggggtagaatatctcgtataggtaggtaagacaacgttggaaggggctcactgattacaaatcggacctcatttccaagattgcctgaggatttcttctgctgggggagggggatatcgtgtatataaggcctcatctacttccctctacatgataataataataccgaatcaatcgatccatcattgttgactctatttctacggcctaccagtgttaaaccactcaccttgaagtgaacaagtca

SEQ ID NO: 3 - DNA, 1491 bp, Penicillium chrysogenum
cttgatatgtggagcctgcgtagattgacatataatctgacaatgcaagaatgcagacgtgcatggtcacgcgatgggatggatatcgacccaaaacaaggctaagccaattcggctagtcgcagctcagcagttatcgtccaattaaatggtcaattggacgtgcaaggatatggaggtcaatgcagagcaagtgggcaaggttgtctgggaatgtggagtcatgcgaagcggtgaacatctgccaaatctggggttaatgagttttgaccacgccaaagtgaaagtgagcaagtcgacgagattgatgcatctcggttggggcatgaatcacccgtgaaggtcgagcgcatcactcctgccagcccatcgagggacgcagtggacgaggtgagatcgggtggtcgttcacatcaaaacacgtgggttcccacagacgataagctgggtatccaccaagactcgagcgatagatacaatcagtacgcggtaaaagagacatgcggtttgtactgtggaatatcgatatattgtaattggaaggaagagcaatggggaggctgaatgagaccaacagaggccaatagcctccaacaaggacactcagcttgatagtcacgaggttcaaaggattcccgggacaccgacggtccaagaagtccggaaacagttttgtaaaaaacgagtcgggtaattttgaatggcaacatcacatggggccaatcacaccatcgatggatgtctcatttccacaagtggcaatttcaccttgagcctaatcagaaagccccgcatctttgaccaggtgcagttgcctgtgattgggtcccaggccatgtgtgagatatcaaagtccagctatctcacctcggttatcagtcattcttcaatgtgataggctggatgagatggccaagagacagggttacaatggaagttaccagccaagcctcccgacgcggggagaaactcggccaaaccgtgggccaagatataccaagacgcactaacccgacactaagtaacaccaagtggatatcaatcccaggggtctcagcccacagtagctcattgattggttgatccaaattcagacatccattagcctctcgggatagttatcttttacttttccaacaaagcgcgacttccaaggttttgcttatcgacggggaaagtggtcacggccggatgaggccgaagctcacttccaaggttaatctcgtaaaagcactaaaagcactaagacgcagggtcttcgccggctattgacggtttttgccaagtccgccgtccgccacgtcccaatccagacattgccccgggattctggaatgagagaagcgagactcttggaggcactaaacaggtacgctaaatcacgccatggaccatataattgcttcacctcgccctcccacaagttttcttttgcttttgctttcctgctgtgtgtttcatctctcacatctctggattccagaaccgcca

SEQ ID NO: 4 - DNA, 1349 bp, Penicillium chrysogenum
tccatcgccgggtcgcctcttgtctttttgatttacctagataggtatgatgtgatatttgcaacataggtgacgtcattttagtttactggaacatcgccagcaaagtgggattatagagatttgaaacgagagggaatgttgatccatttgaagggtatcatcgtgatcgataatggcaggtggttgacaccgggttggacagcatgttcaacgctacgcggttgatgaggctcaaagggaggaccctgttacctgcacagggtgacaggggcaaagtctatgtggatacaccaaagtatcagactatgattctacagttctatagcaatatagagctatagagctaaaaggagaagtatatccgagacataagttgcaacacagaatagaatgcccttagtacattagatcggagaagatgtttcagattcagccaagacaagaaaagaaaaaaaagatgtttttctaaggggaaataaaagagaggtgagttaagtagaagatttagttccacatgtaaccgtccccccagttgggccgactaccaccgttgcactcaagggggtcattgagtatgtatgggtacattgggaatgtgctcggtattatattcttgtgttcttatattctaaatgatttggagtattattctatctaaggaatatattagatctggactgtatgactatcaactattaacactattacatacactattaacctacgtaggttctataaagtaaagtaatacatatacaacagcacatgtaactccactccacccattactacaggtgtggtgtatgttgtatatgggatataaactgtatatgaatggtatggtatgagaatggcctttttatgaactgttaatgactcttctaaagtgactgtacaatagccaaaagcaataacctccgtagatttatttcctgtcacctgcacaacatagacataaattaccgttacagtaccatacccatgcacattgcgacatcgcaagggcttttcccccagttgcacccgcggtgacactgtcaactcaagaatgggccaatgttgatccatgcttagggtcaacacccttcttttccttctcttctcttcttctctctccttttcactttctccgggtggggaattactgcttcactcccaccgatccactcaaaacacaactccagtgggagtgtcacatcggcttgcaccccccccccacccccccaaaccttagcttataacttcagattgttcttcttttatctcatctgctccatgatcaatcatatttcaacatagtgaacttcgatcccggttcgcgacctcccctctcctcccttcctcga

SEQ ID NO: 5 - DNA, 1442 bp, Penicillium chrysogenum
ttccccttctggtgattgaccccaaccccagggcccacccccgctaaatgtctcccttgggtggtggacccgtttcccagcccgttcttctcctacgtcgccagtgctttttttggtttgaaaaacaacagtcgattcggaagtagcatcttgactctattgccactcggagtggtcaccatatgcaggtacaatatttatcgagacactcggaggatgctaacctttcggtaacttgctctgtggtaaggccgggagatatcaagtgaccattaagctcaatgagactctgcaagcgcccggggtatctttgaggcctttatttgacccatcagagctactatgtacaacgttgtacctccataagccctgtattggataataagttagctcatagggttatggtctcctgagtttgggccctttgcagaacctcttattatttgaggaggcaaattatggcgctgtgtgtgaccccaagcaatttcaatcaagatatatttatacctagttatgtcgtattattgtcattattgtcattattgtcatttcatattaattagacaatgcgtcggaccaagtcccaatgtggtatagttcgatgctacgctctctcgggccctaccggggtccaatcttccccggattttcctcaaaacagagactatggggtttgccactttatgatgcttgtcaataattctgtcatacattcatgcaattgttttggtctgcatatcttatttgattgctttgtagcaaacttcaaggttctagcccgtgggaacgctgcacaaccgctacatgtacgaagtactgcaacttgtaccgtaccgccattcaggccgctaagagttctagcccgacacaggggaaattaaagtctgtgatgctattcgaccgcatgacaacttacgactaagaagtgtactaccaacattgccactggtccagtcttttcggtggcttgctcgcttgtctggtctagtctcgacttggcgatgcgggcatacgtacgccgtacgagacaatagaccaaccccgatcctcgctccaatggtcaatcggctgcgggaacccagaatccggcgtcagccttcaggcttggccatgcaatgagcatccggggttctaatccgtggtaattacggatctgagtcaaccgcggccttggcctactttcggtggattgtctctcattaagcatgcggagacatgtgatggtcccacgcgccaacctttgagtgcaacccttgcccacaagggtttctccagcggggaggtggagtgtaaaggaccggcaaagcttataaaaaggccatctcccaagcccagacctggctcttcttctcaactcatccgcactcgctatacatacttgactgtcttttccggcacgccgttcataatatcttctattttttagtcaatccatcaagatg

SEQ ID NO: 6 - DNA, 18 bp, artificial
  forward primer
caaggccgaccacagctt

SEQ ID NO: 7 - DNA, 19 bp, Artificial
  reverse primer
gccgagacggttgacgaat

SEQ ID NO: 8 - DNA, 21 bp, Artificial
  forward primer
ggtgatgaggcacagtcgaag

SEQ ID NO: 9 - DNA, 23 bp, Artificial
  reverse primer
agctcgttgtagaaggtgtggtg

SEQ ID NO: 10 - DNA, 27 bp, Artificial
  primer
gtcaaagcttgtgtacttaccaatggc

SEQ ID NO: 11 - DNA, 29 bp, Artificial
  primer
agtcatgcatgctgaatgaaggcgggaga

SEQ ID NO: 12 - DNA, 19 bp, Artificial
  forward primer
ttcatgccagtccagcgtt

SEQ ID NO: 13 - DNA, 18 bp, Artificial
  reverse primer
cgaccgcaaaccgaagtc

SEQ ID NO: 14 - DNA, 20 bp, Artificial
  forward primer
aacaacattgcgctcgatga

SEQ ID NO: 15 - DNA, 20 bp, Artificial
  reverse primer
ccttggaaaccgtgttcgtt

SEQ ID NO: 16 - DNA, 27 bp, Artificial
  primer
gtcaaagcttccggctcggatctcgtc

SEQ ID NO: 17 - DNA, 29 bp, Artificial
  primer
gagggcatgctgacttgttcacttcaagg

SEQ ID NO: 18 - DNA, 22 bp, Artificial
  forward primer
tgaagttcgacgaggactgatg

SEQ ID NO: 19 - DNA, 21 bp, Artificial
  reverse primer
cgcttgagacgtcaggttgtt

SEQ ID NO: 20 - DNA, 27 bp, Artificial
  primer
gtcaaagcttgatatgtggagcctgcg

SEQ ID NO: 21 - DNA, 29 bp, Artificial
  primer
tcatgcatgctggcggttctggaatccag

SEQ ID NO: 22 - DNA, 21 bp, Artificial
  forward primer
ttgtcctggtctttccccatt

SEQ ID NO: 23 - DNA, 20 bp, Artificial
  reverse primer
ccaggcctaccgtaccattg

SEQ ID NO: 24 - DNA, 31 bp, Artificial
  primer
gtcaaagcttgaattccatcgccgggtcgcc

SEQ ID NO: 25 - DNA, 29 bp, Artificial
  primer
tcatgcatgctcgaggaagggaggagagg

SEQ ID NO: 26 - DNA, 19 bp, Artificial
  forward primer
ctgcagagcgatggttgct

SEQ ID NO: 27 - DNA, 23 bp, Artificial
  reverse primer
ttggagacagagacttggtcctt

SEQ ID NO: 28 - DNA, 25 bp, Artificial
  primer
gtcagaattccccttctggtgattg

SEQ ID NO: 29 - DNA, 25 bp, Artificial
  primer
gctgatgcatcttgatggattgact

SEQ ID NO: 30 - DNA, 28 bp, Artificial
  primer
cacttaagcttcccgtattatccccatc

SEQ ID NO: 31 - DNA, 31 bp, Artificial
  primer
ccctatgcatgcgtgactgattaaacaaggg